(19) United States
(12) Patent Application Publication
(10) Pub. No.: US 2022/0015998 A1
(43) Pub. Date: Jan. 20, 2022

(54) TOPICAL COMPOSITION COMPRISING VIABLE MICROORGANISMS

(71) Applicant: Lactobio A/S, Copenhagen O (DK)

(72) Inventors: Charlotte Vedel, Copenhagen O (DK); Ida Blomquist Jørgensen, Copenhagen O (DK); Søren Kjærulff, Copenhagen O (DK)

(73) Assignee: Lactobio A/S, Copenhagen O (DK)

(21) Appl. No.: 17/311,260

(22) PCT Filed: Dec. 19, 2019
(86) PCT No.: PCT/EP2019/086169

§ 371 Date: Jun. 4, 2021

(30) Foreign Application Priority Data
Dec. 21, 2018 (DK) PA 2018 01052

(51) Int. Cl. A61K 8/11; A61K 8/99; A61K 9/00; A61K 9/107; A61K 35/747

(52) U.S. Cl. CPC A61K 8/11; A61K 8/99; A61K 9/0014; A61Q 19/00; A61K 35/747; A61K 2800/56; A61K 9/1075

(57) ABSTRACT

The present invention relates to a microcapsule comprising a fat-based coating surrounding a composition providing an encapsulated composition, the encapsulated composition comprising a viable microorganism, and a water content below 5% (w/w).

### TECHNICAL FIELD OF THE INVENTION

The present invention relates to a topical composition comprising microorganisms. In particular the present invention relates to a topical composition comprising viable microorganisms which composition is stable and may be activated when applied on skin.

### BACKGROUND OF THE INVENTION

There is considerable interest in the use of probiotic bacteria. Probiotics are live microorganisms that confer health benefits to the host when administered at adequate levels. However, to exert these benefits, the microorganisms must remain viable during the processing and storage of the product. Considerable amount of research has been done to stabilize probiotics when used for oral consumption to ensure resistance to gastrointestinal fluids. Because probiotics are sensitive to a number of factors, including the presence of oxygen and acidic media, microencapsulation has been studied as a method of increasing the viability of probiotic cells.

Microencapsulation of probiotics is a process where probiotic microorganisms is surrounded by a polymeric membrane, protecting them and, in certain cases, allowing their release under specific conditions. The techniques commonly applied to encapsulate probiotics are extrusion, atomization or spray drying, emulsion, coacervation and immobilization in fat polysaccharides or starch granules. Polysaccharides, such as alginate, gellan, K-carrageenan, and starch are the most commonly used materials in the microencapsulation of bifidobacteria and lactobacilli.

Thus, microencapsulation of microorganisms is well known in the art, however, these techniques are not developed for topical use and the microcapsules traditionally produced are designed to be dissolved in the intestinal tract releasing the microorganisms in the gut rather than on the skin. When prior art microcapsules are applied on the skin, the conditions on the skin will not dissolve the capsules and release the live microorganisms. WO18002248 disclose a concept of formulating microorganisms in a 2-compartment system, protecting the microorganisms of the inner core compartment from the ingredients in the outer compartment once the content of both compartments is combined, this microencapsulation is for topical use, however, still this encapsulation comprises microcapsules of a size touchable to the skin and which needs to be rubbed into the skin to break the capsules. The capsules not broken by friction will then not release the viable microorganisms to the surface of the skin.

The use of viable probiotics for topical application is very limited and most products are based on lysates (inactivated dead bacteria) of the probiotic strain (WO8200093) to overcome the problems of maintaining viability of the microorganisms in the topical composition.

Hence, an improved formulation solving the above mentioned problems with the prior art, comprising live probiotic strains in oils, emulsions, lotions and the like for topical application on the skin of mammals would be desirable. In particular an improved formulation comprising viable probiotic strains, which is stable, and which is capable of being activated when applied on the skin would be advantageous.

The present invention relates to a topical composition comprising microorganisms. In particular the present invention relates to a topical composition comprising viable microorganisms which composition has a long shelf life and is stable and may be activated when applied on skin.

### SUMMARY OF THE INVENTION

Thus, an object of the present invention relates to a topical composition comprising viable microorganisms.

In particular, it is an object of the present invention to provide a topical composition that solves the above mentioned problems of the prior art with the presence of viable microorganisms, long shelf life and high stability and the effect of being activated when applied to the skin of a mammal.

Thus, one aspect of the invention relates to a microcapsule comprising a fat-based coating surrounding a composition providing an encapsulated composition, the encapsulated composition comprising a viable microorganism, and a water content below 5% (w/w).

Another aspect of the present invention relates to a topical composition comprising the microcapsule according to the present invention.

Yet another aspect of the present invention relates to a composition comprising the microcapsule according to the present invention, or the topical composition according to the present invention, for use as a medicament.

Still another aspect of the present invention relates to a composition comprising the microcapsule according to the present invention, or the topical composition according to the present invention, for the treatment, alleviation and/or prophylaxis of a skin disorder.

An even further aspect of the present invention relates to a method for providing a microcapsule according to the present invention, wherein the method comprises the steps of:
(i) providing a composition comprising a viable microorganism;
(ii) adding a fat to the composition comprising the viable microorganism, providing a fat mixed microorganism;
(iii) mixing the fat mixed microorganism providing the microcapsule according to the present invention.

### BRIEF DESCRIPTION OF THE FIGURES

FIG. 1 shows a close up of a particle having a centre (1) comprising the viable freeze-dried lactic acid bacteria surrounded by a fat-based coating (2),

FIG. 2 shows the fat encapsulated freeze-dried lactic acid bacteria (LAB) according to the present invention after 3 months of storage.

The present invention will now be described in more detail in the following.

### DETAILED DESCRIPTION OF THE INVENTION

The inventors of the present invention found that the use of probiotics in topical formulations may have a huge potential if viability of the microorganisms could be maintained in the formulations. However, it was found that topical formulations like creams, lotions, mists inherently contain a high degree of water, i.e. in order to be suitably formulated into a cream, foam, lotion, ointment etc. Evidently, the presence of such high degrees of water in these formulations, poses a problem for the storage of probiotics in their metabolically inactive condition. A second problem occurring in such aqueous topical formulations, may be that these formulations generally contain agents, which are not compatible with the survival of microorganisms; such as preservatives, surfactants, emulsifiers and other ingredients in order to protect such formulations against the growth of unwanted microorganisms as well as for forming stable emulsions. However, these agents of course will also form a major problem in the formulation of beneficial microorganisms.

Thus, topical formulations and products for pharmaceutical or cosmetic purposes are developed to have a long shelf life and to be stable towards contamination and spoilage caused by microorganisms is highly desirable.

Hence, it is an object of the present invention to provide a system or formulation allowing for long-term storage of microorganisms, in particular viable microorganisms, which does not substantially harm such microorganisms upon use thereof and which does release the viable microorganisms when applied on the skin.

A preferred aspect of the present invention relates to a microcapsule comprising a fat-based coating surrounding a composition providing an encapsulated composition, the encapsulated composition comprising a viable microorganism, and a water content below 5% (w/w).

It was surprisingly found that coating the microorganisms with a fat, e.g. a butterfat, being solid at storage temperature but melting at skin temperature (when applied to the skin) allowed for both a stability of the viable microorganisms as well as a release and activation of the microorganisms immediately when applied to skin.

In the context of the present invention the term “immediately” relates to the release and activation of the microorganisms from the fat-based coating at the time of applying the encapsulated microorganism to the skin. The melting may be caused by the skin temperature and the heat generated from friction when applying the encapsulated microorganism to the skin.

In the context of the present invention the term “coating” relates to the fat layer surrounding the viable microorganism. The coating according to the present invention is surrounding the viable microorganism completely separating the viable microorganisms inside the coating from the exterior environment outside the coating. The coating according to the present invention is characterized by being solid or partly solid at room temperature and dissolves when applied to skin of a mammal.

In the context the term “embedding” or “embedded”, which is used interchangeably, relates to the dispersion of the coated microorganisms in a hydrophobic phase and/or hydrophilic phase according to the present invention.

In an embodiment of the present invention the encapsulated composition may comprise below 5% (w/w) water; such as below 4% (w/w); e.g. below 3% (w/w); such as below 2% (w/w); e.g. below 1% (w/w); such as below 0.5% (w/w); e.g. below 0.1% (w/w); such as below 0.05% (w/w); e.g. below 0.01% (w/w).

The water content may be measured by the Karl Fisher analysis which is known to the person skilled in the art.

The microcapsule and/or the composition according to the present invention can be made delicious with long term stability, as the low water activity (Aw) fat-based coating protects the dry viable cultures from moisture. It is an added advantage that the fat, e.g. butter fat, used for the fat-based coating may have a softening effect on the skin.

In a further embodiment of the present invention the microcapsule comprises at least 10² CFU/g; such as at least 10³ CFU/g; e.g. at least 10⁴ CFU/g; such as at least 10⁵ CFU/g; e.g. at least 10⁶ CFU/g; such as at least 10⁷ CFU/g; e.g. at least 10⁸ CFU/g; such as at least 10′⁹ CFU/g; e.g. at least 10¹² CFU/g; such as in the range of 10²-10¹⁴ CFU/g; e.g. in the range of 10³-10¹² CFU/g; such as in the range of 10⁴-10¹⁰ CFU/g; e.g. in the range of 10⁵-10⁹ CFU/g; such as in the range of 10⁶-10¹⁰ CFU/g; e.g. in the range of 10⁷-10⁹ CFU/g.

In order to achieve proper distribution when applied to the skin and providing of fast activation and release of the viable microorganisms the particle size of the microcapsules may be important.

As mentioned previously, the fat-based coating according to the present invention may be 35 characterized by being solid or partly solid at room temperature and dissolves when applied to skin of a mammal. Thus, in an embodiment of the present invention the fat-based coating has a melting temperature in the range of 25-37° C.; such as a melting temperature in the range of 28-36° C.; e.g. a melting temperature in the range of 29-35° C.; such as a melting temperature in the range of 31-34° C.

In a further embodiment of the present invention the fat-based coating may comprise a triglyceride with a fatty acid composition of at least 30% oleic acid (C18:1) and at least 30% Stearic acid (C18:0).

Preferably, the fat-based coating may comprise a fat selected from shea butter fat, illipe fat, mango butter fat, kanya butter and cocoa butter fat or any combinations thereof.

Even more preferably the fat-based coating may be shea butter fat.

The encapsulated composition according to the present invention may be embedded in a hydrophobic phase.

In an embodiment of the present invention the hydrophobic phase may be an oil. The oil may be a combination of two or more oils, such as three or more oils, e.g. 4 or more oils, such as 5 or more oils.

In an embodiment of the present invention the encapsulated composition and/or the encapsulated composition embedded in a hydrophobic phase may be emulsified in a hydrophilic phase.

The structure and function of most microorganisms may be dependent upon their aqueous environment. Therefore, changes to their aqueous environment resulting from freezing and/or drying processes can often have drastic consequences for a biological material.

Furthermore, freeze-drying combines the stresses due to both freezing and drying. The freezing step of this process can have undesirable side effects, such as the denaturation of proteins and enzymes, and rupture of cells. These effects result from mechanical, chemical, and osmotic stresses induced by crystallization of ice in these materials. As a result, the viability of the microorganism upon rehydration is lost either in its entirety, or to such a significant extent that the microorganism is no longer viable.

In order to improve stability of the encapsulated composition and maintain the stability of the viable microorganisms, the viable microorganism may be dried.

Drying of the viable microorganisms may be performed by various methods such as by freeze-drying; ambient air drying; vacuum drying or spray drying. Preferably, the viable microorganism is freeze-dried.

To prevent or reduce the adverse effects upon reconstitution or rehydration, protective agents, such as cryoprotectants or lyoprotectants (freeze-drying) may be used. Such protective agents must in order to be effective in the present invention be non-toxic to the microorganism at the concentrations encountered during preservation, and they must interact favourably with water and with the microorganism. Various protective agents have been used in the art, with varying degrees of success.

In an embodiment of the present invention the encapsulated composition may comprise a protective agent. The protective agent may be a cryoprotectant, a lyoprotectant or a combination hereof.

In an embodiment of the present invention the encapsulated composition comprises a protective agent selected from the group consisting of proteins, such as fish proteins; polymers; skim milk; glycerol; dimethyl sulfoxide; and polyhydroxy compounds. The encapsulated composition comprises a protective agent which is preferably a polyhydroxy compounds.

In a further embodiment of the present invention the polyhydroxy compounds may be selected from sugars or carbohydrates.

In another embodiment of the present invention the polyhydroxy compounds may be selected from monosaccharides, disaccharides or polysaccharides.

In yet an embodiment of the present invention the polyhydroxy compounds may be selected from maltose; lactose; sucrose; trehalose; skim milk powder; dextran; dextrose; peptone; glutamate; poly ethylene glycol (PEG); or any combination hereof. Preferably, the combination of polyhydroxy compounds comprises a combination of sucrose and trehalose.

The encapsulated composition according to the present invention comprises in the range of 10-95% (w/w) protective agent relative to the encapsulated composition, such as in the range of 20-80% (w/w); e.g. in the range of 30-70% (w/w); such as in the range of 40-60% (w/w); e.g. in the range of 45-55% (w/w).

The amount of protective agent, such as polyhydroxy compound, can be determined from the amount present in the protectant agent and/or from the amount present in a composition comprising the viable microorganism. Alternatively, the amount of protective agent, such as polyhydroxy compound, in the encapsulated composition can be determined by analytical methods known in the art, such as column chromatography.

In an embodiment of the present invention the encapsulated composition may further comprise a salt, such as a phosphate salt, e.g. sodium phosphate. The sodium phosphate may preferably be sodium hydrogen phosphate; disodium hydrogen phosphate; or a combination of the two.

A preferred aspect of the present invention relates to a topical composition comprising the microcapsule according to the present invention.

In an embodiment of the present invention the topical composition may be an emulsion comprising a hydrophilic phase and a hydrophobic phase. The hydrophobic phase comprises the microcapsule according to the present invention.

The hydrophilic phase may constitute 5-75% (w/w) of the topical composition; such as 10-50% (w/w); e.g. 15-40% (w/w); such as 20-30% (w/w).

In yet an embodiment of the present invention the topical composition comprises 5-75% (w/w) water; such as 10-50% (w/w) water; e.g. 15-40% (w/w) water; such as 20-30% (w/w) water.

The topical composition according to the present invention comprises a preservative; a surfactant; and/or an emulsifier. Preferably, the preservative; a surfactant; and/or an emulsifier is found in the oil or in the hydrophilic phase, preferably in the hydrophilic phase.

In the context of the present invention the term “fat-based coating” and “fat” relates to a substance comprising primarily carbon and hydrogen atoms and which is hydrophobic and soluble in organic solvents and insoluble in water.

The fat-based coatings according to the present invention may preferably be substantially solid at room temperature and melt on the skin just under body temperature. “Room temperature,” as used herein, refers to indoor temperatures commonly, typically on the order of about 20° C. A typical coating fat will have a melting point of about 25° C. to about 37° C.

In a preferred embodiment the fat is solid or partly solid at temperatures below 25° C.

In an embodiment the fat-based coating or the fat may be selected from shea butter fat, illipe fat, cocoa butter fat, mango butter fat, kanya butter fat. Preferably, the fat-based coating or fat may be characterized by constituting a substantially continuous fat phase.

It is preferred that the microorganism may be a probiotic culture product. It is further preferred that the probiotic culture products disclosed herein remain essentially dry, and that they contain no more than a trace of water. The use of substantial quantities of water in processing is typically incompatible with the coating fats and the product stability.

The fat encapsulated composition comprising viable microorganisms can be used for topical application directly as a fat composition.

The fat encapsulated composition comprising viable microorganisms can be further processed (embedded) into a liquid oil wherein the fat encapsulated composition comprising microorganisms is in a concentration from 0.1 to 95% of the embedded composition.

The fat encapsulated composition comprising viable probiotics can be further processed into an emulsion comprising a hydrophilic phase from 0.1 to 95% of the emulsion.

The oil comprising fat encapsulated composition comprising viable probiotics (the embedded composition) can be further processed into an emulsion comprising a hydrophilic phase from 0.1 to 95% of the emulsion.

In one preferred embodiment of the invention the topical composition is an emulsion consisting of a hydrophilic phase and a hydrophobic phase wherein the hydrophobic phase comprises fat encapsulated composition comprising viable microorganisms.

Emulsifiers can be used to stabilize the topical composition and/or topical emulsions, emulsifiers for topical emulsions are known in the art and can be selected from fractionated lecithins enriched in either phosphatidyl choline or phosphatidyl ethanolamine, or both; mono and diglycerides thereof; monosodium phosphate derivatives of mono and diglycerides of edible fats or oils; lactylated fatty acid esters of glycerol and propylene glycol; hydroxylated lecithins; polyglycerol esters of fatty acids; propylene glycol; mono and diester of fats and fatty acids; DATEM (diacetyl tartaric acid esters of mono and diglycerides); PGPR (polyglycerol polyricinoleate); polysorbate 20, 40, 60, 65 and 80; sorbitan monostearate; sorbitan tristearate, oat extract; and the like. The emulsifier is not limited by this list.

The present invention may relate to live or viable microorganisms including any bacteria, archaea, phages, viruses, yeast or fungi or any combinations thereof.

In an embodiment of the present invention the viable microorganism may be a probiotic microorganism.

Examples of suitable probiotic microorganisms may include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcusfaecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus.

The probiotic microorganisms according to the present invention may preferably be in powdered form, dried form; or in spore form (for microorganisms which form spores). In an embodiment of the present invention the probiotic microorganism or the viable microorganism may be a strain of lactic acid bacteria (LAB). Particularly, the probiotic microorganism or the viable microorganism may be a strain of lactic acid bacteria (LAB) selected from the genera Lactobacillus, Leuconostoc, Bifidobacterium, Pediococcus, Lactococcus, Streptococcus, Aerococcus, Carnobacterium, Enterococcus, Oenococcus, Sporolactobacillus, Tetragenococcus, Vagococcus, and Weissella.

The probiotic microorganism or the viable microorganism may include the families Lactobacillaceae, Aerococcaceae, Carnobacteriaceae, Enterococcaceae, Leuconostocaceae and Streptococcaceae. This family of microorganisms are considered non-pathogenic and are used as probiotic bacteria in general to improve gastrointestinal flora and in the treatment of gastrointestinal symptoms. Lactobacilli are important in particular in the food industry, where they play an important role in the area of “functional food.” In the past, the Bifidobacterium bifidum species was classified with the lactobacilli (Lactobacillus bifidum), but according to today's understanding, this species is not closely related phylogenetically to that order. However, it is still considered to be a lactic acid bacterium with regard to the metabolism. Lactic acid bacteria are classified as non-pathogenic.

Lactic acid bacteria do also have a potential use in topical skin care and topical pharmaceutical products and is used in the prior art as dead in-activated cells or instable formulations.

The present invention relates to stabilization of any viable microorganism, such as a bacteria, in a topical composition. The bacteria are preferably selected among the genera Lactobacillus, Leuconostoc, Bifidobacterium, Pediococcus, Lactococcus, Streptococcus Aerococcus, Carnobacterium, Enterococcus, Oenococcus, Sporolactobacillus, Tetragenococcus, Vagococcus, and Weisella. Lactobacillus may be preferred.

The preferred microorganisms may be a bacteria. Preferably the bacteria may be a probiotic bacterium. In an embodiment of the present invention the probiotic bacteria may preferably be selected from the group comprising Lactococcus lactis, Lactobacillus rhamnosus, Lactobacillus plantarum, Lactobacillus helveticus, Lactobacillus jensenii, Lactobacillus acidophilus, Lactobacillus bulgaricus, Lactobacillus amylovorus, Lactobacillus amylolyticus, Lactobacillus alimentarius, Lactobacillus aviaries, Lactobacillus delbrueckii, Lactobacillus diolivorans, Lactobacillus farciminis, Lactobacillus gallinarum, Lactobacillus casei, Lactobacillus crispatus, Lactobacillus gasseri, Lactobacillus johnsonii, Lactobacillus hilgardii, Lactobacillus kefiranofaciens, Lactobacillus kefiri, Lactobacillus mucosae, Lactobacillus panis, Lactobacillus paraplantarum, Lactobacillus pontis, Lactobacillus sakei, Lactobacillus saliverius, Lactobacillus sanfraciscensis, Lactobacillus paracasei, Lactobacillus pentosus, Lactobacillus cellobiosus, Lactobacillus collinoides, Lactobacillus coryniformis, Lactobacillus crispatus, Lactobacillus curvatus, Lactobacillus brevis, Lactobacillus buchneri, Lactobacillus fructivorans, Lactobacillus hilgardii, Lactobacillus fermentum, Lactobacillus reuteri, Lactobacillus ingluviei, Weissella viridescens, Bifidobacterium bifidum, Bifidobacterium adolescentis, Bifidobacterium breve, Bifidobacterium longum, Bifidobacterium animalis, Camobacterium divergens, Corynebacterium glutamicum, Leuconostoc citreum, Leuconostoc lactis, Leuconostoc mesenteroides, Leuconostoc pseudomesenteroides, Oenococcus oeni, Pasteuria nishizawae, Pediococcus acidilactici, Pediococcus dextrinicus, Pediococcus parvulus, Pediococcus pentosaceus, Probionibacterium freudenreichii, Probionibacterium acidipropoinici, Streptococcus thermophilus, Bacillus amyloliquefaciens, Bacillus atrophaeus, Bacillus dausii, Bacillus coagulans, Bacillus flexus, Bacillus fusiformis, Bacillus lentus, Bacillus licheniformis, Bacillus megaterium, Bacillus mojavensis, Bacillus pumilus, Bacillus smithii, Bacillus subtilis, Bacillus vallismortis, Geobacillus stearothermophilus or mutants thereof.

In another aspect of the present invention the probiotic microorganism may be selected from the genera related to the natural healthy skin microbiome including genera Probionibacterium, Cutibacterium, Staphylococcus, Corynebacterium, Malassezia, Aspergillus, Cryptococcus, Rhodotorula, and/or Epicoccum.

In an embodiment of the present invention the probiotic strain may be Staphylococcus epidermidis, Staphylococcus hominis, Cutibacterium acnes (Probionibacterium acnes) or any combinations thereof.

In an embodiment of the present invention the probiotic strain may be a Gram-negative bacteria.

In a further embodiment of the present invention the probiotic strain may be an ammonia oxidizing bacteria.

In yet an embodiment of the present invention the probiotic strain is Nitrosomonas eutropha.

In one preferred embodiment of the present invention the encapsulated composition comprises at least one strain (preferably a viable strain) selected from the group consisting of Weissella viridescens LB10G (DSM 32906), Lactobacillus plantarum LB113R (DSM 32907), Lactobacillus plantarum LB244R (DSM 32996), Lactobacillus paracasei LB116R (DSM 32908), Lactobacillus paracasei LB28R (DSM 32994), Lactobacillus brevis LB152G 25 (DSM 32995), Lactobacillus plantarum LB316R (DSM 33091), Leuconostoc mesenteriodes LB349R (DSM 33093), Lactobacillus plantarum LB356R (DSM 33094), Lactobacillus plantarum LB312R (DSM 33098), and Leuconostoc mesenteroides LB276R (DSM 32997) or mutant strains.

In a preferred embodiment of the present invention the fat encapsulated composition comprising a viable microorganism selected from the list, but is not restricted to: Bifidobacterium lactis DSM10140, B. lactis LKM512, B. lactis DSM 20451, Bifidobacterium bifidum BB-225, Bifidobacterium adolescentis BB-102, Bifidobacterium breve BB-308, Bifidobacterium longum BB-536 from Zaidanhojin Nihon Bifizusukin Senta (Japan Bifidus Bacteria Center), Bifidobacterium NCIMB 41675 described in EP2823822. Bifidobacterium bifidum BB-225, Bifidobacterium adolescentis BB-102, Bifidobacterium breve BB-308, Bifidobacterium lactis HN019 (Howaru) available from DuPont Nutrition Biosciences ApS, Bifidobacterium lactis DN 173 010 available from Groupe Danone, Bifidobacterium lactis Bb-12 available from Chr. Hansen A/S, Bifidobacterium lactis 420 available from DuPont Nutrition Biosciences ApS, Bifidobacterium breve Bb-03, B. lactis HN019, B. lactis BI-04, B. lactis Bi-07 available from DuPont Nutrition Biosciences ApS, Bifidobacterium bifidum Bb-02, Bifidobacterium bifidum Bb-06, Bifidobacterium longum KC-1 and Bifidobacterium longum 913 (DuPont Nutrition Biosciences ApS), Bifidobacterium breve M-16V (Morinaga) and/or a Lactobacillus having a probiotic effect and may be any of the following strains; Lactobacillus rhamnosus LGG (Chr. Hansen), Lactobacillus acidophilus NCFM (DuPont Nutrition Biosciences ApS), Lactobacillus bulgaricus 1260 (DuPont Nutrition Biosciences ApS), Lactobacillus paracasei Lpc-37 (DuPont Nutrition Biosciences ApS), Lactobacillus rhamnosus HN001 (Howaru) available from DuPont Nutrition Biosciences ApS, Streptococcus thermophilus 715 and Streptococcus thermophilus ST21 available from DuPont Nutrition Biosciences ApS, Lactobacillus paracasei subsp. paracasei CRL431 (ATCC 55544), Lactobacillus paracasei strain F-19 from Medipharm, Inc. L. paracasei LAFTI L26 (DSM Food Specialties, the Netherlands) and L. paracasei CRL 431 (Chr. Hansen), Lactobacillus acidophilus PTA-4797, L. salivarius Ls-33 and L. curvatus 853 (DuPont Nutrition Biosciences ApS). Lactobacillus casei ssp. rhamnosus LC705 is described in FI Patent 92498, Valio Oy. Lactobacillus rhamnosus GG (LGG) (ATCC 53103) is described in U.S. Pat. No. 5,032,399 and Lactobacillus rhamnosus LC705 (DSM 7061), Propionic acid bacterium eg. Propionibacterium freudenreichii ssp. shermanii PJS (DSM 7067) described in greater details in FI Patent 92498, Valio Oy, Nitrosomonas eutropha D23 (ABiome), Staphylococcus hominis strains A9, C2, AMT2, AMT3, AMT4-C2, AMT4-GI, and/or AMT4-D12. (all from Matrisys Bioscience), Staphylococcus epidermidis strains M034, M038, All, AMT1, AMT5-05, and/or AMT5-G6 (all from Matrisys Bioscience), L. plantarum YUN-V2.0 (BCCM LMG P-29456), L. pentosus YUN-V1.0 (BCCN LMG P-29455), L. rhamnosus YUN-S1.0 (BCCM LMG P-2961) and/or any combinations hereof.

The use of viable probiotics for topical application today is very limited and products are either unstable or based on lysates of in-activated probiotic strains to overcome the problems of maintaining stability and viability of the microorganisms in the topical composition. The problems observed with traditional formulations when formulating live probiotic strains in oils, serums, emulsions, lotions and the like for topical application on the skin of mammals are lack of viability and stability.

Compositions for topical applications are typically to be stable for about a month at room temperature, this is a major problem for maintaining viability of live probiotic in skin care products.

In an embodiment of the present invention the topical composition may be stabile for at least 2 months when stored at 25° C.; such as for at least 3 months when stored at 25° C.; e.g. for at least 4 months when stored at 25° C.; such as for at least 5 months when stored at 25° C.; e.g. for at least 6 months when stored at 25° C.; such as for at least 7 months when stored at 25° C.; e.g. for at least 8 months when stored at 25° C.; such as for at least 9 months when stored at 25° C.; e.g. for at least 10 months when stored at 25° C.; such as for at least 11 months when stored at 25° C.

Another problem observed may be activation of the probiotic strain when applied on the skin of a mammal. If the probiotic strain is microencapsulated, e.g. by following the procedures used for stabilization of probiotics for oral consumption, then the microcapsules are designed to protect the live probiotic strain in the gastrointestinal fluids and will thus not dissolve on the skin surface. Therefore, the probiotic strain will not be released from the encapsulation and thereby not able to establish a binding, a metabolism or colonization of the probiotic strain on the skin surface.

The present invention solves the problem of stabilization by encapsulating live probiotic strains in a solid fat-based coating, which may be used in a composition for topical use.

The present invention relates to live microorganisms for topical application to the skin of a mammal. The skin may be the outer covering of the body and is the largest organ of the integumentary system. The skin has up to seven layers of ectodermal tissue and guards the underlying muscles, bones, ligaments and internal organs.

The inventors of the present invention surprisingly found that encapsulating the viable microorganisms in a fat-based coating according to the present invention resulted in maintenance of viability and facilitated the probiotic effect on the skin surface.

It will be understood that in the following, preferred embodiments referred to in relation to one broad aspect of the invention are equally applicable to each of the other broad aspects of the present invention described above. It will be further understood that, unless the context dictates otherwise, the preferred embodiments described below may be combined. When used herein, the term topical includes references to formulations that are adapted for application to body surfaces (in particular to the skin or mucous membranes). Mucous membranes that may be mentioned in this respect include the mucosa of the vagina, the penis, the urethra, the bladder, the anus, the nose and the ear.

The present invention further provides a therapeutic composition for the treatment or prevention of an skin disorder, comprising a therapeutically-effective concentration of one or more live species or strains within a pharmaceutically-acceptable carrier suitable for administration to the skin of a mammal and/or a topical administration on the skin or mucous membranes of a mammal, wherein said probiotic strain possesses the ability to maintain viable in the composition at room temperature and be released when applied to the skin surface.

In another aspect, the invention relates to a composition comprising a pharmaceutically or cosmetically acceptable vehicle or excipient.

The composition according to the present invention may be present in solid, liquid, viscous form or as skin cream. The composition is preferably in the form of an emulsion. More preferable the composition is a cream or lotion.

In one preferred embodiment the invention relates to a topical composition for the skin of either humans or animals.

In an embodiment of the present invention the composition is a lotion, serum, oil, or emulsion comprising fat encapsulated microorganisms.

The composition according to the present invention may advantageously further comprise other probiotics, prebiotics, antimicrobials, antibiotics or other active antibacterial substances and/or may preferably also contain one or more of the following substances selected from antioxidants, vitamins, coenzymes, fatty acids, amino acids and cofactors.

In an embodiment of the present invention, the composition is a topical pharmaceutical, veterinary, cosmetic or skin care product.

The composition may preferably comprise one or more thickeners, wherein the thickener may be selected from cellulose ether, polysaccharides, selected from the group comprising xanthan gum, gelatin, highly dispersed silicon dioxide, starch, carrageans, alginates, tragacanth, agar, gum arabic, pectin or polyvinyl esters.

Furthermore, the composition may also comprise builders, enzymes, electrolytes, pH regulators, thickeners, antioxidants, prebiotics, optical brighteners, graying inhibits, foam regulators and/or coloring agents.

The composition may comprise one or more prebiotic sources for the probiotic strain to restore metabolism on the skin surface.

In a preferred embodiment of the invention the composition comprising at least one live probiotic strain for use in the treatment of a skin disorder.

A further preferred aspect of the present invention relates to a composition comprising the microcapsule according to the present invention, or the topical composition according to according to the present invention, for use as a medicament.

An even further preferred aspect of the present invention relates to a composition comprising the microcapsule according to the present invention, or the topical composition according to anyone the present invention, for the treatment, alleviation and/or prophylaxis of a skin disorder.

In the context of the present invention the term “skin disorder” and “skin disease” may be used interchangeably and vary greatly in symptoms and severity. Skin disorder and skin disease may be temporary or permanent and may be painless or painful. Some have situational causes, while others may be genetic. Some skin conditions are minor, and others can be life-threatening.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of disease, stabilized (i.e., not worsening) state of disease, prevention of disease, delay or slowing of disease progression, and/or amelioration or palliation of the disease state. The decrease can be a 10 percent, 20 percent, 30 percent, 40 percent, 50 percent, 60 percent, 70 percent, 80 percent, 90 percent, 95 percent, 98 percent or 99 percent decrease in severity of complications or symptoms.

In addition, the invention relates to compositions containing the fat encapsulated composition comprising microorganisms, in particular for use in treating skin disorders or skin diseases, in products for topical use.

In an embodiment of the present invention the skin disease may be selected from the group of skin diseases comprising psoriasis, atopic dermatitis, dry skin, sensitive skin, acne prone skin, hyperpigmented skin, aged skin, allergy, eczema, rashes, UV-irritated skin, photodamaged skin, detergent irritated skin (including irritation caused by enzymes used in washing detergents and sodium lauryl sulphate), Rosacea, and thinning skin (e.g. skin from the elderly and children).

In a further embodiment the composition according to the present invention may be used for cosmetic skin care.

In an even further embodiment of the present invention, the composition comprising a fat encapsulated composition comprising at least one probiotic microorganism according to the present invention may be used on the skin of patients with inflammatory skin diseases.

In yet an embodiment of the present invention, the composition comprising a fat encapsulated composition comprising at least one probiotic microorganism may be used on the skin of patients with inflammatory skin diseases, wherein the fat coating is a fat with anti-inflammatory effects on skin.

In an embodiment of the present invention, the composition comprising a fat encapsulated composition comprising at least one probiotic microorganism may be used on the skin of patients with inflammatory skin diseases, wherein the fat coating is shea (shea nut) butter fat.

In a further embodiment of the present invention the skin disorder may be associated with atopic dermatitis, eczema, impetigo, acnes, burns, diaper rash, wounds.

The composition of the present invention may be used alleviating; curatively or prophylactically, for example, as a probiotic treatment of the skin or mucous membranes.

In an embodiment of the present invention the composition may be a topical composition. PH of the composition of the present invention may be between approx. 3 and approx. 8. More preferable a pH between 4-7 and even more preferable a pH between 4.5-6.5.

Natural butter fats comprise natural antioxidants, in an embodiment of the present invention further antioxidants may be incorporated into the composition. Antioxidants may preferably be Vitamin E (0.25 to 2.5 wt %) and/or Rosemary extract (0.1 to 0.75 wt %).

A “reduction” in viability may be “statistically significant” as compared to the viability determined at the time of formulating the composition. reduction in viability may be measured as a log reduction and may include a log reduction of 5 or less; such as 4.5 or less; e.g. 4 or less; such as 3.5 or less; e.g. 3 or less; such as 2.5 or less; e.g. 2 or less; such as 1.5 or less; e.g. 1 or less; such as 0.5 or less; e.g. 0.1 or less. “Viability” of microorganisms is measured as Colony Forming Units CFU/ml.

A “reduction” in viability of microorganisms may be determined as the difference in CFU/ml as compared to the CFU/ml at the time of formulating the composition.

The microorganisms according to the present invention may be in isolated or purified form. In the present context the term “isolated” means that the microorganism may be derived from their culture medium including their natural medium, for example. The term “purified” is not restricted to absolute purity.

The microorganisms may advantageously be present in viable spray-dried and/or lyophilized form.

Preferably, the probiotic strain may be used as a live isolated microorganism in a dried form. Suitable methods for cryoprotection has been described previously.

In the context of the present invention the terms “live” and “viable” may be used interchangeable.

In a preferred embodiment of the invention the microorganism may be used as a viable isolated lyophilized microorganism.

The microorganism may be present in the composition in an amount by weight of 0.001% (w/w) to 20% (w/w), preferably 0.005% (w/w) to 10% (w/w), especially preferably 0.01% (w/w) to 5% (w/w).

An embodiment of the present invention involves the administration of from approximately 1×10³ to 1×10¹⁴ CFU of viable bacteria per gram of the composition, more preferably from approximately 1×10⁴ to 1×10¹⁰, and most preferably from approximately 1×10⁵ to 1×10⁹ CFU of viable bacteria per gram of composition.

In yet an embodiment of the present invention the dosage of live probiotic microorganisms in the composition may be above approximately 1×10⁴ CFU of viable bacteria per gram of the composition, preferably above approximately 1×10⁵ and more preferably from approximately 1×10⁶ CFU of viable bacteria per gram of composition and more preferably from approximately 1×10⁷ CFU of viable bacteria per gram of composition.

It was surprisingly found that the microorganisms according to the present invention may be able to activate on the skin and re-establish metabolic activity despite the presence of other microbial species in the skin microbiome.

It will be clear to those skilled in the art that here, as well as in all the statements of range given in the present invention, characterized by such terms as “about” or “approximately,” that the precise numerical range need not be indicated with expressions such as “about” or “approx.” or “approximately,” but instead even minor deviations up or down with regard to the number indicated are still within the scope of the present invention. In the present context the minor deviation relates to a deviation of 5% or less; such as a deviation of 4% or less; e.g. a deviation of 3% or less; such as a deviation of 2% or less; e.g. a deviation of 1% or less; such as a deviation of 0.5% or less; e.g. a deviation of 0.1% or less.

A “mammal” include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; piglets; sows; poultry; turkeys; broilers; minks; goats; cattle; horses; and non-human primates such as apes and monkeys.

Preferable the composition may be for topical use on the human skin.

An “effective amount” depends upon the context in which it is being applied. In the context of administering a composition according of the present invention, an effective amount may be the addition of a number of viable microorganisms determined as CFU/gram which has a probiotic effect on skin.

In an embodiment of the present invention the microcapsule and/or the composition comprising the encapsulated composition comprising the microorganism may comprise a prebiotic compound. “prebiotic compounds” or “prebiotics” are components that increase the growth of specific microorganisms. “Synbiotics” are compositions comprising at least one probiotic and at least one prebiotic compound. Such compositions are understood to encourage the growth of beneficial microorganisms (e.g. the probiotic). Thus, powerful synbiotics are based on a combination of specific strains of probiotic microorganisms with carefully selected prebiotics. They can lead to an important health benefit to a mammal.

Prebiotics refer to chemical products that induce the growth and/or activity of commensal skin microorganisms (e.g., bacteria and fungi) that contribute to the well-being of their host. Prebiotics stimulate the growth and/or activity of advantageous bacteria that colonize the skin. Prebiotics can thus serve as a food source for probiotics. Prebiotics are well known in the art.

In an embodiment of the present invention the prebiotics may be selected from carbohydrates, glucans, alpha-glucans, beta-glucans, mannan-oligosaccharides, inulin, oligofructose, human milk oligosaccharides (HMO), galactooligosaccharides (GOS), lactulose, lactosucrose, galactotriose, fructo-oligosaccaride (FOS), cellobiose, cellodextrins, cylodextrins, maltitol, lactitol, glycosilsucrose, betaine, Vitamin E or a variant thereof (wherein the variants are selected from alfa, beta, gamma, delta tocoferols, tocotrienols and tocomonoenols). Optionally, mannan-oligosaccharides and/or inulin may be preferred.

HMOs may include lacto-N-tetraose, lacto-N-fucopentaose, lacto-N-triose, 3′-sialyllactose, lacto-N-neofucopentaose, sialic acid, L-fucose, 2-fucosyllactose, 6′-sialyllactose, lacto-N-neotetraose and 3-fucosyllactose.

In an embodiment of the present invention at least one of the following prebiotic compounds are used in the topical composition of the invention; lactose, beta-glucans, mannan-oligosaccharides, inulin, oligofructose, galactooligosaccharides (GOS), lactulose, lactosucrose, galactotriose, fructo-oligosaccaride (FOS), cellobiose, cellodextrins, cylodextrins, maltitol, lactitol, glycosilsucrose, betaine, Vitamin E or a variant thereof (wherein the variants are selected from alfa, beta, gamma, delta tocoferols, tocotrienols and tocomonoenols), lacto-N-tetraose, lacto-N-fucopentaose, lacto-N-triose, 3′-sialyllactose, lacto-N-neofucopentaose, sialic acid, 2-fucosyllactose, 6′-sialyllactose, lacto-N-neotetraose and 3-fucosyllactose. Optionally, lactose and/or mannan-oligosaccharides and/or inulin may be preferred.

Fucose, in particular L-fucose, may be preferred, since this compound is believed to strengthen natural defense of skin, stimulate epidermis immune defense and/or prevent and/or treat cutaneous autoimmune disease. In one preferred embodiment of the invention the composition comprises L-fucose and/or D-fucose.

In an embodiment of the present invention the composition further comprises L-fucose and/or D-fucose in a concentration in the composition of 10 mM to 500 mM.

The composition according to the present invention comprising the encapsulated microorganism of the invention may further comprises at least one further probiotic microorganism selected from the group consisting of bacteria, archaea, phages, virus, yeasts or molds.

In an embodiment of the present invention the composition comprising the encapsulated microorganism of the present invention and at least one other strain, wherein the at least one other microorganism may be selected from:

A bifidobacterium may be any bifidobacterium having a probiotic effect, typically strains belonging to the species Bifidobacterium animalis, Bifidobacterium breve, Bifidobacterium infantis, Bifidobacterium lactis, Bifidobacterium longum, Bifidobacterium bifidum and Bifidobacterium adolescentis are used. The one or more live Bifidobacterium lactis strains are selected from, but not restricted to, B. lactis BI-04, B. lactis Bi-07, B. lactis 420, B. lactis DN 173 010, B. lactis HN019, B. lactis Bb-12, B. lactis DR10, B. lactis DSM10140, B. lactis LKM512, B. lactis DSM 20451, Bifidobacterium bifidum BB-225, Bifidobacterium adolescentis BB-102, Bifidobacterium breve BB-308, Bifidobacterium longum BB-536 from Zaidanhojin Nihon Bifizusukin Senta (Japan Bifidus Bacteria Center), Bifidobacterium NCIMB 41675 described in EP2823822. Bifidobacterium bifidum BB-225, Bifidobacterium adolescentis BB-102, Bifidobacterium breve BB-308, Bifidobacterium lactis HN019 (Howaru) available from DuPont Nutrition Biosciences ApS, Bifidobacterium lactis DN 173 010 available from Groupe Danone, Bifidobacterium lactis Bb-12 available from Christian Hansen A/S, Bifidobacterium lactis 420 available from DuPont Nutrition Biosciences ApS, Bifidobacterium breve Bb-03 available from DuPont Nutrition Biosciences ApS, Bifidobacterium bifidum Bb-02, Bifidobacterium bifidum Bb-06, Bifidobacterium longum KC-1 and Bifidobacterium longum 913 (DuPont Nutrition Biosciences ApS), Bifidobacterium breve M-16V (Morinaga) and/or a Lactobacillus having a probiotic effect and may be any of the following strains; Lactobacillus rhamnosus LGG (Chr. Hansen), Lactobacillus acidophilus NCFM (DuPont Nutrition Biosciences ApS), Lactobacillus bulgaricus 1260 (DuPont Nutrition Biosciences ApS), Lactobacillus paracasei Lpc-37 (DuPont Nutrition Biosciences ApS), Lactobacillus rhamnosus HN001 (Howaru) available from DuPont Nutrition Biosciences ApS, Streptococcus thermophilus 715 and Streptococcus thermophilus ST21 available from DuPont Nutrition Biosciences ApS, Lactobacillus paracasei subsp. paracasei CRL431 (ATCC 55544), Lactobacillus paracasei strain F-19 from Medipharm, Inc. L. paracasei LAFTI L26 (DSM Food Specialties, the Netherlands) and L. paracasei CRL 431 (Chr. Hansen), Lactobacillus acidophilus PTA-4797, L. salivarius Ls-33 and L. curvatus 853 (DuPont Nutrition Biosciences ApS). Lactobacillus casei ssp. rhamnosus LC705 is described in FI Patent 92498, Valio Oy. Lactobacillus rhamnosus GG (LGG) (ATCC 53103) is described in U.S. Pat. No. 5,032,399 and Lactobacillus rhamnosus LC705 (DSM 7061), Propionic acid bacterium eg. Propionibacterium freudenreichii ssp. shermanii PJS (DSM 7067) described in greater details in FI Patent 92498, Valio Oy, Nitrosomonas eutropha D23 (ABiome), Staphylococcus hominis strains A9, C2, AMT2, AMT3, AMT4-C2, AMT4-GI, and/or AMT4-D12. (all from Matrisys Bioscience), Staphylococcus epidermidis strains M034, M038, All, AMT1, AMT5-05, and/or AMT5-G6 (all from Matrisys Bioscience, L. plantarum YUN-V2.0 (Yun NV, BCCM LMG P-29456), L. pentosus YUN-V1.0 (BCCN LMG P-29455), L. rhamnosus YUN-S1.0 (BCCM LMG P-2961) and any mixtures thereof.

Preferably, the composition comprising the encapsulated microorganism further comprises at least one strain selected from the group consisting of Weissella viridescens LB10G (DSM 32906), Lactobacillus plantarum LB113R (DSM 32907), Lactobacillus plantarum LB244R (DSM 32996), Lactobacillus paracasei LB116R (DSM 32908), Lactobacillus paracasei LB28R (DSM 32994), Lactobacillus brevis LB152G (DSM 32995), Lactobacillus plantarum LB316R (DSM 33091), Leuconostoc mesenteriodes LB349R (DSM 33093), Lactobacillus plantarum LB356R (DSM 33094), Lactobacillus plantarum LB312R (DSM 33098), and Leuconostoc mesenteroides LB276R (DSM 32997) or mutant strains.

In an embodiment of the present invention the composition may comprise at least one strain selected from the group of lactic acid bacteria being able to improve tight junction integrity, e.g. Lactobacillus acidophilus NCFM (DuPont), Lactobacillus salivarius Ls-33 (DuPont), Bifidobacterium lactis 420 (DuPont), L. acidophilus La-14 (DuPont) or L. rhamnosus LGG (Chr. Hansen).

For obtaining a product that acts both on the surface of the skin and through the dermis/epidermis, an emulsion concept may be provided and the emulsion concept may need to be integrated. An emulsion is a mixture of two or more liquids that are normally immiscible (i.e.: oil and water). Emulsions are part of a more general class of two-phase systems of matter called colloids. Although the terms colloid and emulsion are sometimes used interchangeably, emulsion is used when both the dispersed and the continuous phase are liquid. In an emulsion, one liquid (the dispersed phase) is dispersed in the other liquid (the continuous phase).

In a preferred embodiment of the invention, the fat encapsulated microorganism is suspended in a liquid oil and further incorporated into an emulsion comprising a water phase and an oil phase, wherein the oil phase comprises the microorganisms encapsulated in solid fat.

A “liquid” oil of the invention is an oil being liquid a storage temperature, thus the liquid oil has a melting point below 25° C., such as below 20° C., e.g. below 15° C. In a preferred embodiment of the invention the liquid oil is a vegetable selected from almond oil, hemp oil, CBD oil, cannabis oil Evening prim rose, Borage oil, Almond sweet oil, Rose Hip oil, Jojoba Golden oil, Camomile oil, Calendula oil, Sea buckthorn oil, Jaf flower oil and sesame oil.

The vegetal oil may comprise at least one of: acai, acai berry, almond sweet, aloes vera, andiroba, apricot kernel, arnica, argan, avocado, babassu, boabab, black berry seed, black cumin, black currant seed, blueberry, borage, brazil nut, brocoli seed, buriti, calendula, camellia seed, cannabis oil including CBD and THC, canola, copaiba balsam, cape chestnut (yangu), carrot (Daucus carrota), castor, chardonnay grape, chaulmoogra, cherry Kernel, chia seed, chickweed, coconut, coconut fractionated, cotton seed, comfrey, corn, crambe seed, cranberry seed, cucumber seed, echium seed, egg, evening primrose, emu, flax seed, grape seed, hazelnut, hemp seed, horsechest nut seed, jojoba, karanj seed, kiwi seed, kukuinut, macadamia nut, marula, marshmallow, manketti, meadowfoam, milk thistle seed, moringa, mullein, mustard seed, neem, olive, palm, papaya seed, passionflower seed, peach kernel, peanut, perilla, pomegranate, Pentaclethra macroloba, pumpkin seed, raspberry seed, rice bran, rosehip, St. John's Wort oil, safflower, sea buckthorn pulp, sheabutter oil, sesame roasted, sesame seed, soya been, sunflower, tamanu (Calophyllum Inophyllum), thistle, tomato, turkey red, sangre de drago, walnut, watermelon seed, wheatgerm, Abyssinian, Colza, bees wax, lanolin, linseed, mortierella oil, ongokea, paraffinum liquid, peacan, Pegui, Poppy seed, Pracaxi, rapeseed, soybean, tall, tung, veronica, Wheat germ, yangu seed and any combination thereof.

The solid fat for coating the viable microorganism is characterized by being solid at storage temperature and melting at skin temperature.

Preferably the solid fat is a natural vegetable fat or butter. These fats may be triglycerides and the melting point depends on the specific combination of fatty acids in the triglycerides. Therefore, fats can be either chemically modified or mixed to obtain a mixed fat composition with the property of the invention.

In one embodiment of the present invention a mixed fat or chemical modified fat may be used for coating of a viable microorganism, characterized by the fat having a melting point between 25° C. and 37° C.

According to another embodiment, there is provided a fat preparation comprising: a solid fat oil or a semi-solid fat oil; wherein the solid fat oil or the semi-solid fat oil may comprise a supplement chosen from the group consisting of: plants parts, trees, roots, seeds, kernels, nuts, oils, fatty acids, active ingredient, vegetal oils, avocado, bees wax, animal by-products, capuagu, cocoa, cocoa black, coconut, coffee, Illipe, Kokum, Mango, Murumuru, Palm Kernel, Pistachio, Shea, Shealoe, Soya, Tucuma, Ucuuba, Nilotica Shea, Sal (Shorea robusta), Tallow (Adeps bovis) and any combination thereof resulting in a melting point of the fat composition between 25° C. and 37° C.

In an embodiment of the present invention the solid fat may be a triglyceride wherein the fatty acid composition of the triglyceride comprises oleic acid (C18:1) and Stearic acid (C18:0).

The fatty acids of the triglyceride may comprise of at least 30% oleic acid (C18:1) and at least 30% Stearic acid (C18:0).

In an embodiment of the present invention the solid fat may be selected from cocoa butter fat, illipe butter fat, mango butter fat, kanya butter fat and/or shea butter fat or any combinations thereof.

In an embodiment of the invention the composition comprises at least 10% fat.

In a further embodiment of the invention the composition comprises at least 20% fat.

In yet an embodiment of the invention the composition comprises at least 50% fat.

In an even further embodiment of the invention the solid fat comprises at least 50% shea butter fat.

In a further embodiment of the invention the solid fat comprises at least 75% shea butter fat.

In yet an embodiment of the invention the solid fat comprises at least 90% shea butter fat.

The composition according to the present invention may be an emulsion comprising a hydrophilic phase and a hydrophobic phase wherein the hydrophobic phase is at least 50% of the composition and wherein the hydrophobic phase comprises fat encapsulated viable microorganisms.

In an embodiment of the present invention the composition may be an emulsion comprising a hydrophilic phase and a hydrophobic phase wherein the hydrophobic phase comprises an oil and a fat, and wherein the ratio of water:oil:fat is 20-60:30-50:5-20.

In a further embodiment of the present invention the composition may be an emulsion consisting of a hydrophilic phase and a hydrophobic phase wherein the hydrophobic phase comprises an oil and a fat, and wherein the ratio of water:oil:fat is 5-20:5-30:50-90.

The probiotic microorganisms according to the present invention may be capable of proliferating and colonizing on and/or in the mammalian skin.

The present invention successfully addresses the shortcomings of the presently known compositions for topical use. Known compositions for topical use are either not able to maintain the viability of the microorganisms or the microorganisms are not able to be activated on the skin surface.

The present invention provides several advantages. In particular, viability of the microorganisms is kept in the composition even at storage at room temperature. The microorganisms activated by the temperature of the skin releasing the microorganisms from the encapsulation to the skin.

According to another embodiment, there is provided a microorganism encapsulated in a fat with a melting point of approximately 25-37° C. The encapsulated microorganism can be further incorporated into a composition. When applied to skin the fat will melt and release the viable microorganism which will be further activated by the moisture of the skin as well as the moisture and any prebiotics in the topical composition.

In a further aspect, this invention provides methods for preparing a topical composition comprising a fat encapsulated microorganism.

The methods can include a step of providing a melted fat composition having a melting point above 25° C.; homogeneously admixing the melted fat with dried viable microorganisms. The fat composition can be heated to its melting point or slightly above to provide a melted fat admixture with the dried microorganism. In a preferred variation, the microorganism is a freeze dried culture. Also, preferably the microorganism is chilled to below 10° C. prior to admixture with the melted fat.

Importantly, the fat composition is low in free moisture (i.e., A_{w} less than 0.4) so as to minimize exposure of the dried viable microorganism to moisture and to avoid activation of the microorganism. The dried microorganism is admixed to the melted fat optionally along with any supplemental soluble ingredients admixed to form a homogenously inoculated melted fat having 103 to 1012 colony forming units per gram.

The fat composition will solidify when chilled, preferably the fat composition is chilled fast to avoid fat crystallization. The fat composition can be used directly as a composition for topical use. The fat composition comprising viable microorganisms can be further processed.

The method can further involve the following step. The melted fat composition comprising the viable microorganisms is chilled and just before solidification homogeneously mixed in an oil. The oil will then contain fat encapsulated viable microorganisms. The oil composition comprising the fat encapsulated microorganisms can be used directly as a composition for topical use.

The oil composition comprising the fat encapsulated microorganisms can be further processed.

The method can further involve the following step. The oil composition comprising the fat encapsulated microorganisms can be admixed with a hydrophilic composition allowing for emulsification, optionally along with any supplemental soluble ingredients. The fat encapsulated microorganisms will stay in the oil phase. The oil phase can be either the continuously phase or the dis-continuously phase of the emulsion. The emulsion will be a topical composition of the invention comprising a fat encapsulated viable microorganism characterized by the fat coating having a melting point between 32 and 37° C.

An aspect according to the present invention relates to a method for providing a microcapsule according to the present invention, wherein the method comprises the steps of:
(i) providing a composition comprising a viable microorganism;
(ii) adding a fat to the composition comprising the viable microorganism, providing a fat mixed microorganism;
(iii) mixing the fat mixed microorganism providing the microcapsule according to the present invention.

Preferably, the composition comprising the viable microorganism provided in step (i) may be subjected to a step of drying before being mixed with the fat (step (ii)). The step of drying may be performed by freeze-drying; ambient air drying; vacuum drying or spray drying. Preferably, the step of drying may be performed by freeze-drying.

The step of drying may be continued until the composition comprising the viable microorganism comprises below 5% (w/w) water; such as below 4% (w/w); e.g. below 3% (w/w); such as below 2% (w/w); e.g. below 1% (w/w); such as below 0.5% (w/w); e.g. below 0.1% (w/w); such as below 0.05% (w/w); e.g. below 0.01% (w/w).

In order to protect the viable microorganisms from being destroyed a protective agent may be added to the composition comprising the viable microorganism before the composition comprising the viable microorganism may be subjected to the step of drying.

The fat added in step (ii) may be melted before being added to the composition comprising the viable microorganism. Preferably, the fat may be melted by heating the fat to a temperature in the range of 35-75° C., such as 37-65° C., e.g. 40-55° C.

In an embodiment of the present invention mixing of the fat mixed microorganism (step (iii)) may be conducted in order to provide an encapsulated composition comprising the viable microorganism. Mixing of the fat mixed microorganism (step (iii)) may be performed by agitation and/or homogenization.

Following mixing of the fat mixed microorganism (step (iii)) the fat may be allowed to solidify after fat mixture has been mixed, preferably by cooling to a temperature below 37° C., such as a temperature below 35° C., e.g. a temperature below 30° C., such as a temperature below 25° C., e.g. a temperature below 20° C., such as a temperature below 15° C., e.g. a temperature below 10° C., such as a temperature below 5° C., e.g. a temperature below 2° C.

In an embodiment of the present invention an oil may be added to the microcapsule provided in step (iii) providing a hydrophobic phase comprising the microcapsule. The oil may be a mixture of oils.

In a further embodiment of the present invention a hydrophilic phase may be admixed with the hydrophobic phase creating an emulsion.

Provided below is an example of a procedure to produce a composition comprising a fat encapsulated microorganism for topical use.

Procedure 1:
Step 1; Melt coating fat
Step 2; Cool to a temperature before solidification of the fat and add a probiotic microorganism
Step 3; Homogenize
Step 4; Cool to solidification

Procedure 2:
Step 1; Melt coating fat
Step 2; Cool to a temperature before solidification of the fat and add a probiotic microorganism and homogenize
Step 3; Add liquid oil and homogenize
Step 4; Cool to solidification

Procedure 3:
Step 1; Melt coating fat
Step 2; Cool to a temperature before solidification of the fat and add a probiotic microorganism and homogenize
Step 3; Add liquid oil and homogenize
Step 4; admix with hydrophilic phase to create an emulsion
Step 5; Cool to solidification

Procedure 4:
Step 1; Melt coating fat
Step 2; Cool to a temperature of approximately 37° C. and add a freeze-dried probiotic microorganism and homogenize
Step 3; Add liquid oil and homogenize
Step 4; admix with hydrophilic phase to create an emulsion
Step 5; Cool to solidification
Step 6; Store in an airtight container

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

Procedure 2:
Step 1; Melt fat
Step 2; Cool to a temperature of approximately 37° C. and add freeze-dried probiotic microorganism and homogenize
Step 3; Add liquid oil and homogenize
Step 4; admix with hydrophilic phase to create an emulsion
Step 5; Cool to solidification

Fats used in step 1 is given in table 1.

TABLE 1
Fats and the major fatty acid composition of the triglyceride.

| | Shea | | Cocoa | | |
|---|---|---|---|---|---|
| Fatty acid | butter | Illipe | butter | Mango | Kanya |
| Arachidic | 1-2% | | 0.1-1.0% | | 0.1% |
| acid (C20:0) | | | | | |
| Linoleic acid | 4-8% | 0-1.2% | 2.6-3.5% | 7.33% | 0.5-0.7% |
| (C18:2) | | | | | |
| Oleic acid | 43-56% | 32-38% | 32.7-34.6% | 46.22% | 48.7-57.6% |
| (C18:1) | | | | | |
| Palmitic acid | 4-8% | 15-19% | 24.1-25.8% | 6.43% | 2.6-3.9% |
| (C16:0) | | | | | |
| Stearic acid | 31-45% | 42-48% | 33.3-37.4% | 37.73% | 38.4-47.1% |
| (C18:0) | | | | | |

Microorganisms used in the example are Lactobacillus rhamnosus LBB (Chr. Hansen), Weissella viridescens LB10G (DSM 32906), Lactobacillus plantarum LB113R (DSM 32907), Lactobacillus plantarum LB244R (DSM 32996), Lactobacillus paracasei LB116R (DSM 32908), Lactobacillus paracasei LB28R (DSM 32994), Lactobacillus brevis LB152G (DSM 32995) and Leuconostoc mesenteroides LB276R (DSM 32997). Microorganisms are freeze-dried viable strains, except from one experiment using viable fresh cultured strains of Leuconostoc mesenteroides LB276R (DSM 32997) and Lactobacillus rhamnosus LBB (Chr. Hansen) grown in MRS media and harvested by centrifugation and tested for viability in the different fats.

The following oils are used in step 3;
Sample 1: Almond oil
Sample 2: Borage oil
Sample 3: Almond sweet oil
Sample 4: Rose Hip oil
Sample 5: Jojoba Golden oil
Sample 6: Camomile oil
Sample 7: Calendula oil
Sample 8: Sea buckthorn oil
Sample 9: Jafflower Evening prim rose oil
Sample 10: Sesame oil

The following additives are used:
Triaconta nyl
Bees wax
Poly sorbate 40, 60 or 80
Lecithin
Glycerin
tocopherol
Inulin
Lactose
L-fucose
Alpha-glucan oligosaccharide
Additives can be added in step 1, 3 or 4.

Viability of the strains was determined in the different fats and oils. All tested strains were found to be stable in the 5 fats, especially in shea butter, none of the freeze dried strains lost their viability when tested after 3 days at 25° C.

Some of the oils e.g. Sea buckthorn oil and Rose Hip oil had antimicrobial activity and to maintain viability of the strains in these oils it is important to embed the viable strain in fat before mixing into the oil.

### Example 2

The following compositions were produced following the procedure 4 above.

Composition 1:
Shea butter fat: 10 g
Freeze dried microorganisms: 1 g (correspond to approximately 10⁹ CFU/ml of final composition
Almond oil: 50 g
Bees wax: 6 g
Glycerin: 3.48 g
Polysorbate 80: 1.16 g
Water: 45.36 g

Composition 2:
Shea butter fat: 30 g
Freeze dried microorganisms: 1 g (correspond to approximately 10⁹ CFU/ml of final composition
Almond oil: 50 g
Bees wax: 6 g
Glycerin: 3.48 g
Polysorbate 80: 1.16 g
Water: 25.36 g

Composition 3:
Shea butter fat: 80 g
Freeze dried microorganisms: 1 g (correspond to approximately 10⁹ CFU/ml of final composition
Sesame oil: 10 g
Bees wax: 6 g
Glycerin: 3.48 g
Polysorbate 80: 1.16 g
Acidified Water (pH 5): 10 g
Tocopherol: 0.1 g

Composition 4:
Shea butter fat: 40 g
Freeze dried microorganisms: 1 g (correspond to approximately 10⁹ CFU/ml of final composition
Almond oil: 10 g
Jojoba oil: 30 g
Bees wax: 6 g
Glycerin: 3.48 g
Polysorbate 80: 1.16 g
Water: 20 g
Inulin: 5 g

Composition 5:
Kanya butter fat: 50 g
Freeze dried microorganisms: 1 g (correspond to approximately 10₉ CFU/ml of final composition
Jojoba oil: 15 g
Triacontanyl: 6 g
Glycerin: 3.48 g
Lecithin: 1 g
Water: 35 g
Lactose: 5 g
Alpha-glucan oligosaccharide: 1 g

Composition 6:
Shea butter fat: 50 g
Cocoa butter fat: 20 g
Freeze dried microorganisms: 1 g (correspond to approximately 10⁹ CFU/ml of final composition
Almond sweet oil: 15 g
Lecithin: 1 g
Water: 20 g
Lactose: 5 g
L-fucose

Composition 7:
Shea butter fat: 30 g
Viable harvested microorganisms: corresponding to approximately 10⁹ CFU/ml of final composition
Almond oil: 50 g
Bees wax: 6 g
Glycerin: 3.48 g
Polysorbate 80: 1.16 g
Water: 25.36 g
Inulin: 5 g

Compositions was stored in air-tight bottles at 20° C. and 25° C. CFU/ml was determined after 1, 2, 5, 7, 14, 30, 60 and 90 days.

All compositions were stable with a log reduction of less than 0.5 in the full test period and the strains maintained viability. Except from composition 7 comprising viable harvested strains not being freeze dried before coating the strain in the fat. For Composition 7 the viability drops after 1 week to approximately 10⁷ CFU/ml and after 60 days the viability was 10⁴-10⁶ CFU/ml. After 90 days no viability in composition 7 was observed whereas all other compositions having freeze-dried encapsulated microorganisms had still maintained viability.

### Example 3

The water content of the freeze-dried/lyophilized microorganism was determined by Karl Fischer titrations using Karl Fischer Aquastar reagents from Merck, Water standard owen kits (Merck 1.88054) and following the standard analysis for water determination provided with the kit in the interval of <0.1% to >5% (w/w). The effect of water content in the freeze-dried composition was evaluated by fat encapsulation of the freeze-dried viable microorganism using the polyhydroxy compounds trehalose (Sigma Aldrich T9449) and sucrose (Sigma-Aldrich 84097) as cryoprotectants. Lactobacillus plantarum LB244R was grown overnight in 1 L MRS at 37° C. and harvested by centrifugation creating a concentrated aqueous cell mass. The cryoprotectants were used as approximately 50% of the aqueous concentrated cell mass of Lactobacillus plantarum LB244R (LAB) the preservation medium contained 200 g of each cryoprotectant and 3.5 g NaH₂PO₄, H₂O, 7.1 g Na₂HPO₄ and 400 mL deionized water was added to the resuspended cell mass (approximately 3% (w/v) cell mass).

The feed suspension was stored in an ice bath for about 30 min prior to use. Each of two freeze-dry bottles were filled with 250 mL of feed suspension. The feed suspensions were frozen quickly by rotating the bottles in dry ice and connected to a freeze drier (Lyph-Locke 6L, Labconco) operated at 950 Pa and 55° C., for 15 min, 45 min, 2 hours, 6 hours and 24 hours (repeated as doublets). The freeze-dried samples were analyzed immediately after for water content, and a fat encapsulation was made following procedure 1 described above.

The fat composition used for encapsulation was:
Shea butter fat (Natura-Tec soft organic shea refined): 10 g
Freeze dried LAB: 1 g
Almond oil (Natura-Tec sweet almond oil—refined): 5 g
Jojoba of (Natura-Tec Jojoba oil refined) I: 5 g
Bees wax (KahlWax organic beeswax 8139): 1 g
Glycerin (Merck 1295607): 1 g

Fat encapsulated freeze-dried LAB was stored at 25° C. and tested for viability at the following times: 0, 7 days, 21 days.

| Water content before | | | |
|---|---|---|---|
| fat encapsulation | CFU/g fat encapsulated freeze-dried LAB | | |
| determined by KF | (stored at 25° C.) | | |
| titration | T = 0 | T = 7 days | T = 21 days |
| 12.7% | 2 × 10⁹ | 0 | 0 |
| 8.2% | 8 × 10⁹ | <10 | 0 |
| 4.9% | 1 × 10¹⁰ | 2 × 10⁵ | 10³ |
| 3.1% | 6 × 10⁹ | 6 × 10⁹ | 5 × 10⁷ |
| 0.09% | 6 × 10⁹ | 3 × 10⁹ | 2 × 10⁹ |
| 11.6% | 4 × 10⁹ | 0 | 0 |
| 7.9% | 2 × 10¹⁰ | 70 | 0 |
| 5.1% | 7 × 10⁹ | 2 × 10⁸ | 100 |
| 2.7% | 3 × 10⁹ | 9 × 10⁸ | 2 × 10⁹ |
| 0.1% | 8 × 10⁸ | 8 × 10⁹ | 2 × 10⁹ |

The lower the water content is in the freeze-dried LAB before fat encapsulation the higher storage stability is obtained.

At low water content a crystallization of the freeze-dried LAB is obtained, and the crystals are encapsulated in the fat composition.

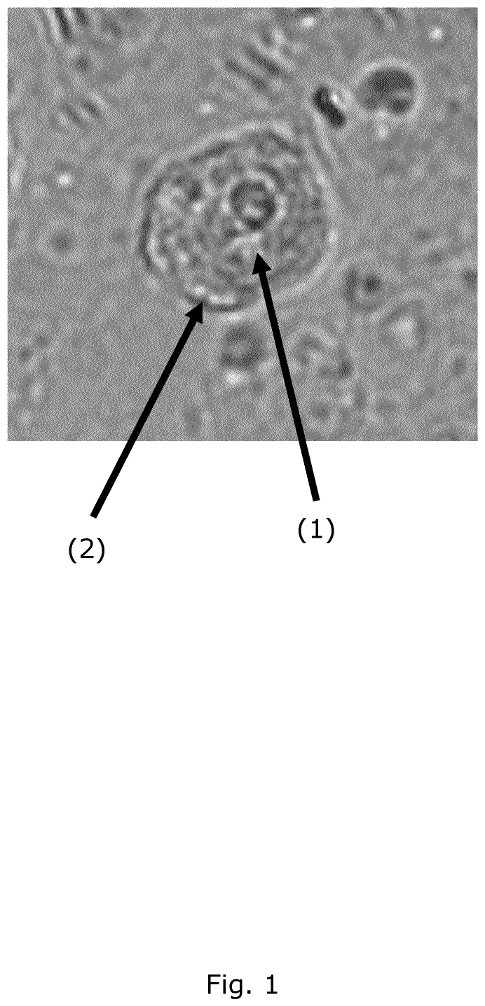

FIG. 1 shows the fat encapsulated freeze dried LAB.

### Example 4

Different cryoprotectants were tested at 2 different water contents of the freeze-dried material.
Skim milk powder for microbiology (Merck 70166)
Peptone (vegetable) (Merck 18332)
Dextran (Sigma-Aldrich S6022)
Dextrose (Sigma-Aldrich G8270)
Glutamate (Sigma-Aldrich G3291)
Trehalose (Sigma Aldrich T9449)
Sucrose (Sigma-Aldrich 84097)
Poly ethylene glycol (PEG) (Sigma-Aldrich 81260)

Lactobacillus plantarum LB244R (LAB) was grown in 200 ml MRS overnight at 37° C., harvested by centrifugation and resuspended in 50 ml of Phosphate buffered Saline PBS, mixed 1:1 with each cryoprotectant and freeze dried as described in Example 3 to two different water contents of approximately 1% and 5% (w/w) determined by KF titration analysis. The cryoprotectants were compared to the stability of a control comprising no cryoprotectants.

After freeze-drying, each sample was encapsulated in a fat composition:
Shea butter fat: 10 g
Freeze dried LAB: 1 g
Almond oil: 10 g

Viability of fat encapsulated freeze-dried LAB was determined semi-quantitative immediately after fat encapsulation and after 14 days storage at 25° C. by plate counting using 15% polysorbate 80 (tween) in water for dilution of the fat encapsulated freeze-dried LAB.

Viability was determined semi-quantitative using the following scale:

| Cryoprotectant | Water content % (w/w) | T = 0 | T = 14 days |
|---|---|---|---|
| Skim milk powder | 5.0 | + + | + + |
| | 0.8 | + + | + + |
| Peptone | 5.3 | + + | + |
| | 1.1 | + + | + + |
| Dextran | 4.8 | + + | + + |
| | 0.9 | + + | + + |
| Dextrose | 5.1 | + + | + + |
| | 1.2 | + + | + + |
| Glutamate | 5.5 | + + | + |
| | 0.7 | + + | + + |
| Trehalose | 4.9 | + + | + + |
| | 1.0 | + + | + + |
| Sucrose | 4.8 | + + | + + |
| | 0.8 | + + | + + |
| PEG | 5.4 | + + | + |
| | 1.2 | + + | + + |
| Control | 5.1 | + + | − |
| | 0.9 | + + | − |

No viability: −
More than 10³ CFU/g +
More than 10⁶ CFU/g + +

Viability of the freeze-dried fat encapsulated LAB was significantly depended on cryoprotectants, in the control without cryoprotectant the fat encapsulated freeze-dried LAB are non-viable (dead) after 14 days. Using cryoprotectants for freeze drying before fat encapsulation was shown to be essential for the viability, however, all the tested cryoprotectants had a significant effect, and only a few cryoprotectants showed a decrease in viability after 14 days and this decrease was observed for the freeze-dried lab with high water content (>5%).

### Example 5

Long term stability was determined for fat encapsulated freeze-dried lactic acid bacteria (LAB) following the procedure described in Example 3.

Lactobacillus plantarum LB244R was freeze dried using dextrose and sucrose as cryoprotectants 1:1. L. plantarum LB244R was grown overnight in 1 L MRS at 37° C. and harvested by centrifugation creating a concentrated aqueous cell mass. The cryoprotectants were used as approximately 50% of the aqueous concentrated cell mass of Lactobacillus plantarum LB244R (LAB) the preservation medium contained 200 g of each cryoprotectant and 3.5 g NaH₂PO₄, H₂O, 7.1 g Na₂HPO₄ and 400 mL deionized water was added to the resuspended cell mass (approximately 3% (w/v) cell mass).

The feed suspension was stored in an ice bath for about 30 min prior to use. Each of two freeze-dry bottles were filled with 250 mL of feed suspension. The feed suspensions were frozen quickly by rotating the bottles in dry ice and connected to a freeze drier (Lyph-Locke 6L, Labconco) operated at 950 Pa and 55° C. and the LAB was freeze dried to a water content of approximately 0.1% and 5% (repeated as doublets). The freeze-dried samples were analyzed immediately after for water content, and a fat encapsulation was made following procedure 1 described above.

Two different fat composition was used for encapsulation:
Fat Composition 1:
Shea butter fat: 10 g
Freeze dried LAB: 1 g
Almond oil: 5 g
Jojoba oil: 10 g
Fat Composition 2:
Shea butter fat: 5 g
Cocoa butter fat: 5 g
Jojoba oil: 10 g

The compositions were stored at 25° C. and viability was fold over time. Viability was determined for each sample once every month for 9 months.

The long-term stability was followed using image analysis. 10 μL of the fat encapsulated freeze-dried LAB were placed in a well of a 96 microtitter plate, melted at 37° C. and 10 μL of MRS growth medium was added on top and growth was followed by image analysis over time in an oCelluScope from BioSense solutions, Denmark. Viability can be determined already after 1 hour by image analysis and detection of number of outgrow from the encapsulated LAB relative to the total number. CFU/g was determined by using a standard curve.

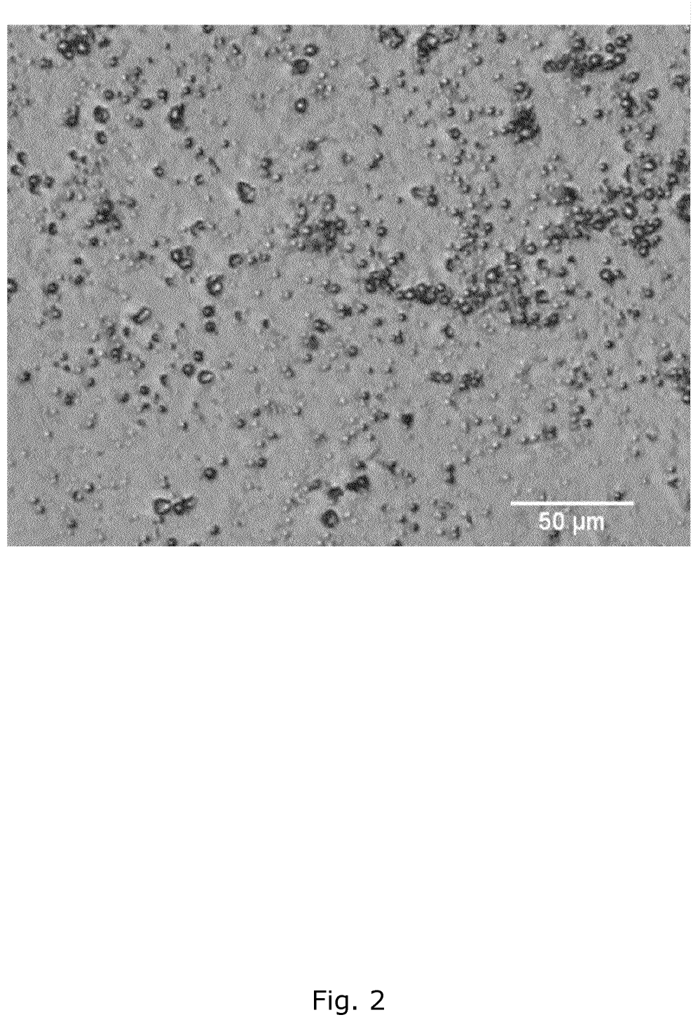

FIG. 2 shows the fat encapsulated freeze dried LAB (fat composition 1) after 3 months of storage.

The viability of the freeze dried fat encapsulated LAB was not significantly different for the two compositions tested, but a significantly difference was seen in the viability depending on the water content of the freeze dried LAB before fat encapsulation.

The low water content (0.1% (w/w)) resulted in a fat encapsulated freeze dried LAB being stable for the entire period of 9 months.

The above description is for the purpose of teaching the person of ordinary skill in the art how to utilize the disclosure provided herein. It is not intended to detail all of those obvious modifications and variations which will become apparent to the skilled worker upon reading the description. It is intended, however, that all such obvious modifications and variations be included within the scope the following claims. The claims are meant to cover the claimed components and steps in any sequence which is effective to meet the objectives there intended, unless the context specifically indicates the contrary.

1. A microcapsule comprising a fat-based coating surrounding a composition providing an encapsulated composition, the encapsulated composition comprising a viable microorganism, and a water content below 5% (w/w).

2. The microcapsule according to claim 1, wherein the encapsulated composition comprises a protective agent, wherein the protective agent is a cryoprotectant or a lyoprotectant.

3. The microcapsule according to claim 1, wherein the encapsulated composition comprises a protective agent, wherein the protective agent is a polyhydroxy compounds, and wherein the polyhydroxy compounds is selected from maltose; lactose; sucrose; trehalose; skim milk powder; dextran; dextrose; peptone; glutamate; poly ethylene glycol (PEG); and a combination hereof.

4. The microcapsule according to claim 1, wherein the encapsulated composition comprises below 5% (w/w) water.

5. The microcapsule according to claim 1, wherein the fat-based coating has a melting temperature in the range of 25−37° C.

6. The microcapsule according to claim 1, wherein the fat-based coating is embedded in a hydrophobic phase, wherein the hydrophobic phase is an oil.

7. The microcapsule according to claim 1, wherein the fat-based coating is emulsified in a hydrophilic phase.

8. The microcapsule according to claim 1, wherein the viable microorganism is dried.

9. A topical composition comprising the microcapsule according to claim 1.

10. The topical composition according to claim 9, wherein the topical composition comprises 5-75% (w/w) water.

11. A composition comprising the microcapsule according to claim 1, for use as a medicament.

12. A composition comprising the microcapsule according to claim 1, for the treatment, alleviation and/or prophylaxis of a skin disorder.

13. A The composition according to claim 1, wherein the skin disorder is selected from psoriasis, atopic dermatitis, dry skin, sensitive skin, acne prone skin, hyperpigmented skin, aged skin, allergy, eczema, rashes, UV-irritated skin, photodamaged skin, detergent irritated skin, Rosacea, and thinning skin.

14. A method for providing a microcapsule according to claim 1, wherein the method comprises the steps of:
(i) providing a composition comprising a viable microorganism;
(ii) adding a fat to the composition comprising the viable microorganism, providing to provide a fat mixed microorganism; and
(iii) mixing the fat mixed microorganism to provide the microcapsule according to claim 1.

15. The method according to claim 14, wherein the composition comprising the viable microorganism provided in step (i) is subjected to a step of drying before being mixed with the fat of step ii.
